(19) United States
(12) Patent Application Publication
(10) Pub. No.: US 2007/0276506 A1
(43) Pub. Date: Nov. 29, 2007

(54) Demineralized osteochondral plug

(75) Inventor: Karen Troxel, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/440,988

(22) Filed: May 25, 2006

(51) Int. Cl. A61F 2/28

(52) U.S. Cl. 623/23.63; 8/94.11

(57) ABSTRACT

A shelf-stable, demineralized, and freeze-dried osteochondral plug comprising cartilage, subchondral bone, and the underlying cancellous bone is provided. Methods of preparing the demineralized osteochondral plug and methods of repairing a defect site are also provided.

### FIELD

The present teachings relate to methods and apparatus for repairing cartilage defects, particularly using an osteochondral plug.

### BACKGROUND

Articular cartilage enables bones to move smoothly relative to one another. Damage to the articular cartilage and the underlying bone can be caused by injury, such as tearing, by excessive wear, or by a lifetime of use. Damage to articular cartilage, particularly of the meniscus and load-bearing regions, causes pain and reduces mobility. Damage to these areas is particularly troubling because damaged articular cartilage does not “heal” completely like other tissues due to the lack of blood and nervous supply in the articular cartilage. Furthermore, when the damage heals naturally, the repair tissue formed is fibrocartilage (generally found in the skin and tendons, for example) which does not have the same biomechanical characteristics as hyaline cartilage (found in the ears and the joints, for example). Accordingly, there is decreased strength and load-bearing abilities of the area.

Medical intervention such as medications, therapy, or surgery can be required to restore proper function to the damaged area. Some of the current procedures for treatment of articular cartilage defects include lavage and debridement, abrasion chondroplasty, microfracture techniques, subchondral drilling, transplantation of periosteal or perichondral grafts, and transplantation of osteochondral autografts or allografts, for example. With respect to osteochondral autografts and allografts, it can be difficult to obtain and store allogeneic implant materials until the time of surgical intervention. Obtaining an implant from the patient can be particularly difficult when there are limited areas from which to obtain an autologous plug or it is not most desired to remove a plug from another area due to potential donor site morbidity, such as with very elderly or feeble patients. Implants from cadavers or other donors are often very scarce and must be implanted into the recipient patient within hours of harvesting to prevent tissue degradation.

Accordingly, there is a need for an osteochondral implant which provides sufficient structural strength and which is storage-stable and allows for the osteochondral implant to be harvested and stored for extended periods of time. It is desirable that the implants be preserved in a state that maximizes integration of the implant into the new defect site and allows for on-demand selection and placement of the osteochondral implant. It is further desirable to provide methods for repairing osteochondral defects while minimizing the risks of donor site morbidity.

### SUMMARY

The present teachings provide a shelf-stable osteochondral plug comprising a layer of cartilage and the underlying bone, where the plug is demineralized and freeze-dried. The underlying bone can comprise the subchondral bone and cancellous bone. The osteochondral plug can have a water content of less than about 6% by weight. The osteochondral plug can be demineralized to have a calcium content of less than about 8% by weight. The osteochondral plug can have a shelf-life of at least about 10 days.

The osteochondral plug can be cylindrical in shape. The osteochondral plug can have a diameter of from about 2 millimeters to about 30 millimeters. The osteochondral plug can have a height of from about 5 millimeters to about 20 millimeters. The osteochondral plug can also include perforations or tunnels in the underlying bone.

A method of preparing an osteochondral plug is provided. Intact donor tissue is removed from a donor site. The intact donor tissue can comprise a region of cartilage and the underlying bone. The donor tissue is demineralized to remove a substantial amount of the content of calcium in the donor tissue. The donor tissue is dehydrated to a final water content of less than about 6% by weight. The dehydration can be selected from methods such as freeze drying, vacuum drying, air drying, temperature flux drying, organic solvents, and combinations thereof.

The demineralization can include reducing the calcium concentration in the donor tissue to less than about 8% by weight. The demineralization can be achieved using a demineralizing agent. The demineralizing agent can be selected from acid-baths, calcium chelator-baths, and combinations thereof. The prepared, shelf-stable implant can be stored for a period of greater than about 10 days.

A method of repairing a cartilage defect site is provided. A shelf-stable demineralized osteochondral plug is hydrated with a hydrating fluid. The defect site is prepared to receive the demineralized osteochondral plug. The demineralized osteochondral plug is press-fit into the defect site. The demineralized osteochondral plug can be shaped by the user. Shaping the demineralized osteochondral plug can include contouring a bone region of the osteochondral plug to the same or larger dimensions than a damaged cartilage region of the defect. The demineralized osteochondral plug can be hydrated with a hydrating fluid. The hydrating fluid can be an aqueous fluid. The aqueous fluids can include water, saline, blood, blood products, platelet concentrate, solutions of growth factors and combinations thereof. The hydration can occur intra-operatively, immediately prior to applying the osteochondral plug to the defect site.

The demineralized osteochondral plug can also include a tissue-health promoting agent such as chondrocytes, undifferentiated cells, differentiation media, growth factors, platelet concentrate, nutrients, bone morphogenic proteins, osteogenic factors, and combinations thereof.

Further areas of applicability of the present teachings will become apparent from the detailed description provided hereinafter. It should be understood that the detailed description and specific examples, while indicating the preferred embodiment of the teachings, are intended for purposes of illustration only and are not intended to limit the scope of the present teachings.

### BRIEF DESCRIPTION OF THE DRAWINGS

The present teachings will become more fully understood from the detailed description and the accompanying drawings, wherein:

FIGS. 1A through 1D depict various osteochondral plugs according to the present teachings;

FIGS. 2A and 2B depict various views of perforated osteochondral plugs according to the present teachings;

FIGS. 3A and 3B depict harvesting an osteochondral plug from an articulating surface according to the present teachings;

FIGS. 4A through 4C depict the process of repairing a defect site using an osteochondral plug according to the present teachings; and

FIG. 5 depicts a cutting apparatus used to harvest the osteochondral plug according to the present teachings.

### DETAILED DESCRIPTION

The following description of the various embodiments is merely exemplary in nature and is in no way intended to limit the present teachings, their application, or uses. It is understood that the present teachings can be used in any cartilage containing area of the body.

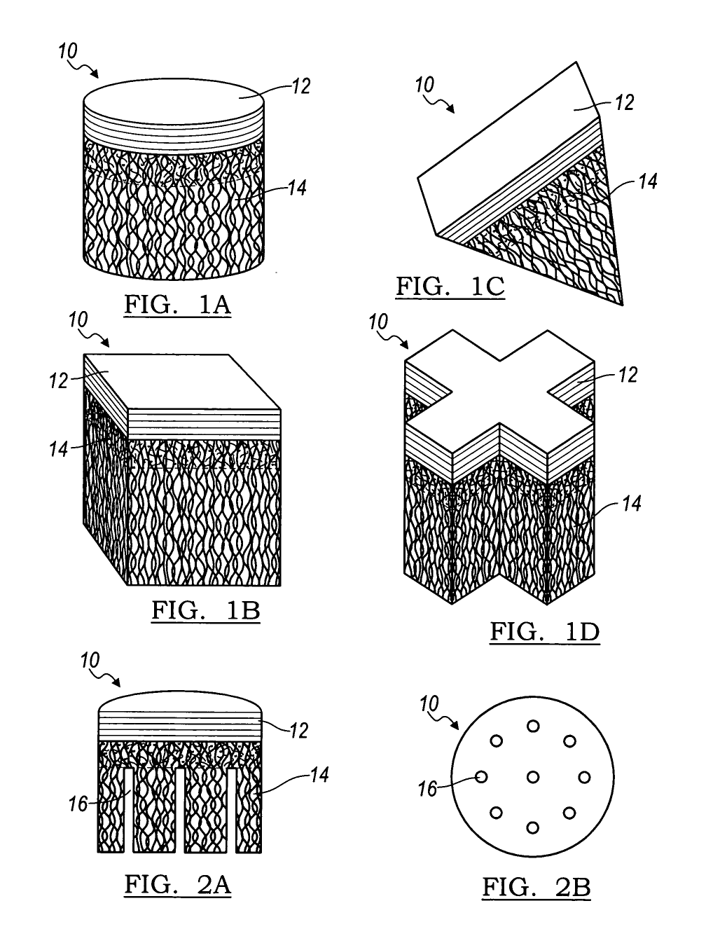

Referring to FIGS. 1A through 1D, the present teachings provide a shelf-stable osteochondral plug 10 comprising a layer of cartilage 12 and the underlying bone 14, where the plug is freeze-dried. As used herein, the term “shelf-stable” refers to the ability of being stored for a period of at least 10 days up to about a year without compositional damage, size degradation or erosions, or a reduction in the load-bearing and other biomechanical properties of the osteochondral plug 10. The shelf-stable osteochondral plug 10, as properly stored, resists moisture damage, bacterial colonization, and can be easily retrieved for on-demand use. The shelf-stable osteochondral plug 10 enhances surgical freedom and flexibility of scheduling because the osteochondral plug 10 is readily available and can be retrieved without requiring planning for harvest of the plug from a cadaveric source. Moreover, the ready-to-use shelf-stable implant allows for intra-operative flexibility should the damaged or degenerative area need a larger implant than anticipated. In such cases, larger osteochondral plugs 10 or multiple osteochondral plugs 10 can be employed. The surgical methods and selections are discussed later herein.

The layer of cartilage 12 is generally a full-thickness layer of cartilage and includes the deep, intermediate, and superficial zones of the cartilage. The underlying bone 14 can comprise the subchondral bone and cancellous bone. The cartilage 12 and the underlying bone 14 are attached via a calcified cartilage layer.

The osteochondral plugs 10 can have a variety of shapes including any regular shape, such as the square plug of FIG. 1B and the triangular plug of FIG. 1C. The osteochondral plug 10 can also have an irregular shape or a free-form shape such as the cross-bar shaped plug depicted in FIG. 1D. The osteochondral plug 10 can be of any implant appropriate size or shape. For example, in various embodiments, the osteochondral plug 10 can be cylindrical in shape. The cylindrical osteochondral plug 10 can have a diameter of from about 2 millimeters to about 30 millimeters. The cylindrical osteochondral plug 10 can have a height of from about 5 millimeters to about 20 millimeters. The osteochondral plugs 10 can be provided in a series of standard sizes for shaping at a later point, as described later herein.

The osteochondral plug 10 is freeze-dried and can have a water content of less than about 6% by weight. As used herein, the term “freeze-dried” or “lyophilization” and variants thereof, refers to the process of isolating the solid component of the osteochondral plug 10 from the water component by freezing the osteochondral plug 10 and evaporating the ice under a vacuum. The freeze-dried osteochondral plug 10 can have a final moisture level of less than about 6% by weight as recommended by the American Association of Tissue Banks.

The osteochondral plug can be demineralized to have a calcium content of less than about 8% by weight. As used herein, the term “demineralized” and variants thereof, means a loss or decrease of the mineral constituents or mineral salts of the individual tissues or bone relative to their natural state. In various embodiments, the demineralized osteochondral plug has a calcium ion concentration of less than about 1% by weight.

FIGS. 2A and 2B, show a side view and a bottom view, respectively, where the underlying bone 14 is optionally perforated to include small tunnels or perforations 16 to increase penetration of the demineralizing agent in the osteochondral plug 10. The perforations 16 are generally contained in the underlying bone 14 and do not extend into calcified cartilage or cartilage 12. The tunnels or perforations 16 can also be used to facilitate uptake of hydrating fluids and/or tissue-health promoting agents as described later herein.

The osteochondral plug 10 can also include a tissue-health promoting agent. Tissue-health promoting agents are useful to expedite the integration of the osteochondral plug 10 into the surrounding tissues. Exemplary tissue-health promoting agents include nutrients, growth factors, bone marrow, undifferentiated cells, chondrogenic factors, osteogenic factors, and the like. In various embodiments, the tissue-health promoting agents can be selectively placed on the osteochondral plug 10. For example, it can be desirable to seed the underlying bone 14 with bone morphogenic proteins. In other embodiments, it can be desirable to seed the underlying bone with undifferentiated cells.

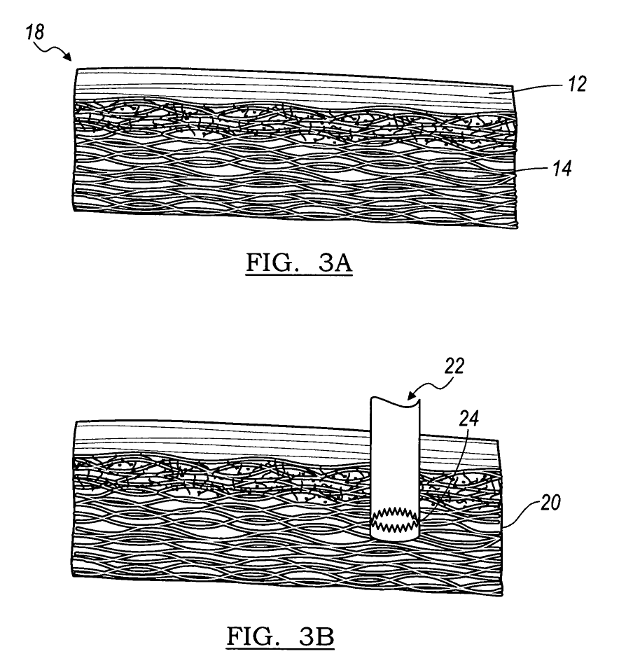

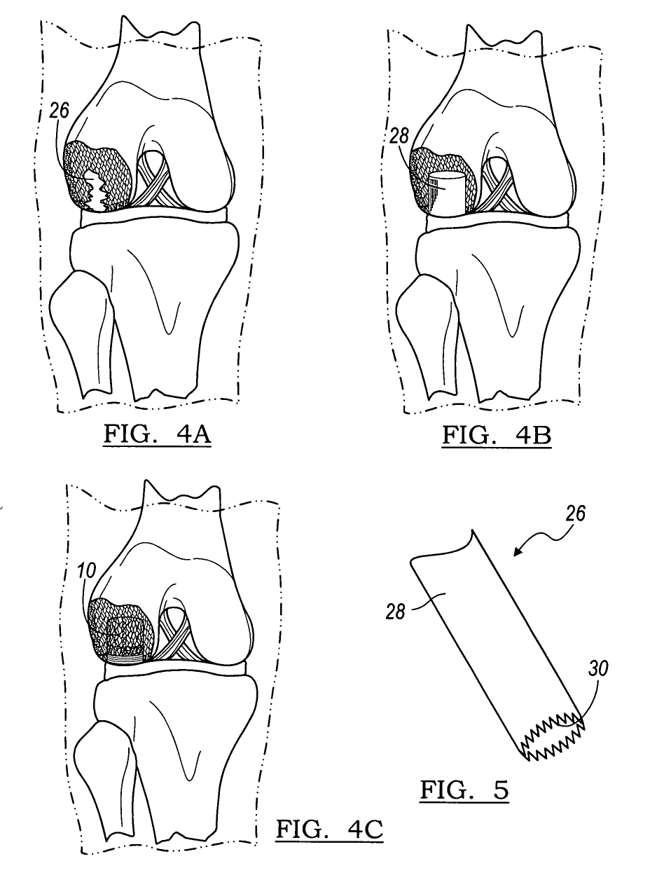

Referring to FIGS. 3A and 3B and FIG. 5, a method of preparing an osteochondral plug 10 is also provided. Intact donor tissue is removed from a donor region or site 18. The intact donor tissue can comprise a region of cartilage 12 and the underlying bone 14. FIGS. 3A and 3B generically depict a donor articular cartilage region. The donor regions 18 can be selected from any articular cartilage region including, but not limited to, femoral condyle, tibial plateau, femoral head, or acetabulum.

A generic cutting instrument 22 depicted in FIG. 5 can be used to retrieve the donor tissue. The generic cutting instrument 22 includes a cutting surface 24. The cutting surface 24 can be a saw or other toothed surface to remove the donor tissue. In use, the donor region 18 is exposed and the cutting instrument 22 is held such that the cutting surface 24 engages the cartilage 12. The cutting instrument 22 is actuated, by turning or electrical means, for example, such that the cutting surface 24 transverses the cartilage 12 and cuts the underlying bone also. When the cutting instrument 22 reaches the appropriate depth, the instrument 22 can be gently rocked back and forth to fracture the cancellous bone to facilitate removal of the donor tissue 20.

The donor tissue 20 is then demineralized to reduce the mineral content of the donor tissue. In various embodiments, demineralization can include reducing the calcium concentration in the donor tissue 20 to less than about 8% by weight. The demineralization can be achieved using a demineralizing agent. The demineralizing agent can be selected from acidification, for example, with acid-baths, chelating processes, for example, with chelator-baths, and combinations thereof.

Suitable acids include, but are not limited to, inorganic acids such as hydrochloric acid or organic acids such as peracetic acid. Chelating agents include, but are not limited to, disodium ethylenediaminetetraacetic acid (Na₂EDTA). Exemplary calcium chelator agents can include any compound having chelating groups to which to adhere the calcium ions, such as 2,2′-Bipyridyl, Dimercaptopropanol, Ethylenediaminotetraacetic acid (EDTA), Ethylenedioxy-diethylene-dinitrilo-tetraacetic acid, Ethylene glycol-bis-(2-aminoethyl ether)-N,N,N′,N′-tetraacetic acid (EGTA), Nitrilotriacetic acid (NTA), Salicylic acid, or Triethanolamine (TEA). In various embodiments EDTA and EGTA can be used to remove the mineral content from the donor tissue.

The time required to demineralize the donor tissue 20 can vary depending on the concentration of acid or chelating agent used, the displacement or flow of the solution and the desired final concentration of calcium in the donor tissue 20. For example, in an embodiment using hydrochloric acid, at an acid concentration of 0.1 to 2.0 N, the donor tissue 20 can be soaked in the acid bath for up to 10 days. The calcium or mineral concentration in the donor tissue 20 can be monitored by measuring the pH of the acid solution using a calcium specific electrode or a standard pH meter. In a preferred embodiment, the acid wash or soak ceases when the calcium concentration of the donor tissue 24 is less than about 8% or less than about 1%.

After demineralization, the pH of the donor tissue 20 is adjusted by removing the acid with a deionized/distilled water wash until the pH of the donor tissue 20 approximates that of the water. It is not outside of the scope of these teachings to expedite the neutralization of the donor tissue 20 using an ionic strength adjuster such as a biocompatible buffer solution. In embodiments having perforations or channels 16, the channels 16 can expedite the neutralization of the donor tissue by facilitating uptake of the wash water or buffer solution.

The demineralized donor tissue 20 can then be lyophilized to a moisture level of less than 6% by weight using standard drying techniques including, but not limited to, freeze drying, vacuum drying, air drying, organic solvent use, evaporation, and combinations thereof. The lyophilization preserves the donor tissue 20 and thereby creates the shelf-stable osteochondral plug 10 that is able to withstand degradation or compromises to the structural integrity of the final osteochondral plug 10. The variety of shapes provided can be preserved and used in case the anticipated needed size of the osteochondral plug 10 varies significantly from what a visual inspection of the defect site mandates.

In various embodiments, the demineralized osteochondral plug 10 can be placed inside of a sterilized dual chamber package. Packaging is preferably durable, flexible, has barrier resistance to moisture, chemicals, grease and bacteria, maintains its integrity upon exposure to low temperatures and is easy to handle in a medical or clinical setting. Suitable packaging materials can include materials selected from the group consisting of thermoplastic films, polyester films, para-aramid fibers, polyethylene fibers, and combinations thereof. In a preferred embodiment, the inner packaging includes a polyester film, such as Mylar® and a polyethylene fiber, such as Tyvek® (both DuPont, Wilmington, Deleware, USA) and the outer compartment is a moisture resistant foil bag made of aluminum and transparent plastic with a Tyvek® Header pouch. Moisture can be drawn from the filled Tyvek Mylar® aluminum/plastic chamber by lyophilizing, vacuum drying, air drying, temperature flux drying, molecular sieve drying and other suitable drying techniques. Preferably, moisture is removed by lyophilizing until the moisture content decreases to about 6% by weight. In various embodiments, the moisture level is less than about 6% by weight. The osteochondral plug 10 is “shelf-stable” in that it will not decompose over an extended period of time, such as 10 days, several months, or up to a year. At the time of surgery, the osteochondral plug 10 can be easily removed from the packaging and is ready for implantation in the defect site. It is understood that multiple osteochondral plugs can be processed simultaneously or the packaging of the plug can vary so long as the conditions in which the osteochondral plug 10 resides limit decomposition of the osteochondral plug 10 and bacterial colonization on the osteochondral plug 10.

A method of repairing a cartilage defect site 26 (or implant site) is provided. Exemplary articular cartilage defects include those caused by trauma, excessive use (such as sports injuries, for example) or diseases, including, but not limited to, osteoarthritis and osteochondrosis dissecans.

The defect site is prepared to receive the osteochondral plug 10. Preparing the defect site 26 can include providing an opening 36 in bone to receive the osteochondral plug 10. The defect site 26 can be shaped by the surgeon to provide the appropriate fit for the osteochondral plug 10. The defect site 26 can be prepared by removing the damaged cartilage with a burr, a curette, or a similar instrument. Once the damaged cartilage is removed down to the calcified cartilage, the size of the defect to prepare as subchondral bone is determined. It may be desirable to contour the subchondral bone region of the defect site to the same or larger dimensions than the cartilage defect region of the defect site. The edges of host cartilage should accommodate a secure press-fit or interference fit of the osteochondral plug 10 in the defect site 26. Bone is removed with a drill or cutting instrument that creates an opening having the same shape, size, and depth as the osteochondral plug 10 or an opening that is slightly smaller than the osteochondral plug 10. The defect site 26 can also be cleaned to provide a healthy tissue base upon which to place the osteochondral plug 10.

The osteochondral plug 10 is shaped. Shaping the osteochondral plug 10 can be achieved by shaving or otherwise trimming the osteochondral plug with a scalpel, surgical drill, or other cutting or resecting devices. In various embodiments, the defect site 26 is prepared such that the opening will provide an interference fit with the osteochondral plug 10.

The osteochondral plug 10 is applied to the implant site or defect site 26 using a press-fit or an interference fit. The osteochondral plug 10 is inserted into the opening 28 through the surrounding cartilage and bone such that the cartilage region 12 of the osteochondral plug 10 is arranged generally flush with the cartilage of the surrounding tissue. A flush osteochondral plug 10 facilitates appropriate articulation in the region. It is understood that the osteochondral plug 10 can be taller, wider, or deeper than the defect site 26 and can protrude above the plane of the surrounding tissue. A slight protrusion (less than about 10%) can allow settling of the osteochondral plug in the defect site 26.

Prior to implantation or after the osteochondral plug 10 is inserted into the opening 28, the demineralized osteochondral plug 10 can be hydrated with a hydrating fluid. The hydrating fluid can be an aqueous solution including, but not limited to, saline, water or a balanced salt solution (e.g., 140 ml NaCl, 5.4 ml KCl, pH 7.6). The aqueous fluids can include blood, blood products, platelet concentrate, solution(s) of growth factor(s), and combinations thereof. The fluids can be ambient fluids from the defect site or extra corpus fluids.

The osteochondral plug 10 can be hydrated by immersing the plug in the hydrating fluid. The hydrating fluid migrates into the pores of the osteochondral plug 10 and the system achieves near complete hydration in minutes, depending on the size of the osteochondral plug 10 and the relative viscosity of the hydration fluid. The hydrated osteochondral plug 10 can be placed directly into the defect site at the surgery or can be placed into a holding dish prior to use in a defect site.

As stated above herein, the osteochondral plug 10 can also include a tissue-health promoting agent. The tissue-health promoting agents can be sprayed or otherwise spread on the osteochondral plug 10 or the osteochondral plug 10 can be soaked in a solution containing the tissue-health promoting agent.

In various embodiments, it can be desirable to incorporate the tissue-health promoting agent into the hydration media. In still other embodiments, the health promoting agent can be incorporated into the osteochondral plug 10 prior to implantation, for example, a nutrient can be placed on the donor tissue 20 such that it is contained in the osteochondral plug 10 prior to arrival in the operating room.

The description of the present teachings is merely exemplary in nature and, thus, variations that do not depart from the gist of the present teachings are intended to be within the scope of the present teachings. Such variations are not to be regarded as a departure from the spirit and scope of the present teachings.

What is claimed is:

1. A shelf-stable osteochondral plug comprising a layer of cartilage and the underlying bone, wherein the plug is demineralized and freeze-dried.

2. The osteochondral plug according to claim 1, wherein the underlying bone comprises the subchondral bone and cancellous bone.

3. The osteochondral plug according to claim 1, wherein the osteochondral plug has a water content of less than about 6% by weight.

4. The osteochondral plug according to claim 1, wherein the osteochondral plug is demineralized to have a calcium content of less than about 8% by weight.

5. The osteochondral plug according to claim 1, wherein the osteochondral plug has a shelf-life of at least about 10 days.

6. The osteochondral plug according to claim 1, wherein the osteochondral plug is cylindrical in shape.

7. The osteochondral plug according to claim 6, wherein the osteochondral plug is from about 2 millimeters to about 30 millimeters in diameter.

8. The osteochondral plug according to claim 6, wherein the osteochondral plug is from about 5 millimeters to about 20 millimeters in height.

9. The osteochondral plug according to claim 1, wherein the underlying bone further comprises perforations.

10. A method of preparing an osteochondral plug comprising:
a. removing from a donor site an intact donor tissue comprising a region of cartilage and the underlying bone;
b. removing a substantial amount of the content of at least one mineral component in the donor tissue; and
c. dehydrating the donor tissue to have a water content of less than about 6% by weight.

11. The method of claim 10, wherein the dehydrating is selected from the group consisting of: freeze drying, vacuum drying, air drying, temperature flux drying, organic solvents, and combinations thereof.

12. The method of claim 11, wherein the dehydrating is freeze drying.

13. The method of claim 10, wherein removing a substantial amount of the content of at least one mineral comprises reducing the calcium concentration in the donor tissue to less than about 8% calcium by weight.

14. The method of claim 13, wherein removing a substantial amount of the content of at least one mineral comprises treating the osteochondral plug with a demineralizing agent.

15. The method of claim 14, wherein the demineralizing agent is selected from the group consisting of acid-baths, calcium chelator-baths, and combinations thereof.

16. The method of claim 10, further comprising storing the osteochondral plug for a period of greater than about 10 days.

17. A method of repairing a cartilage defect site, comprising:
a. hydrating a shelf-stable demineralized osteochondral plug with a hydrating fluid;
b. preparing a defect site to receive the demineralized osteochondral plug; and
c. applying the osteochondral plug to the defect site.

18. The method according to claim 17, wherein preparing the defect site comprises providing an opening in the bone to receive the demineralized osteochondral plug.

19. The method according to claim 17, further comprising shaping the demineralized osteochondral plug to facilitate a press-fit into the defect site.

20. The method according to claim 19, wherein shaping the demineralized osteochondral plug comprises contouring a bone region of the demineralized osteochondral plug to the same or larger dimensions than a damaged cartilage region in the defect site.

21. The method according to claim 17, wherein the hydrating fluid is an aqueous fluid selected from the group consisting of: water, saline, blood, blood products, platelet concentrate, growth factor solution, and combinations thereof.

22. The method according to claim 17, wherein the hydrating occurs immediately prior to the applying of the demineralized osteochondral plug to the defect site.

23. The method according to claim 17, further comprising adding a tissue-health promoting agent to the demineralized osteochondral plug, wherein the tissue-health promoting agent is selected from the group consisting of: chondrocytes, undifferentiated cells, differentiation media, growth factors, platelet concentrate, nutrients, bone morphogenic proteins, osteogenic factors, and combinations thereof.
